(19) United States
(12) Patent Application Publication
(10) Pub. No.: US 2018/0071298 A1
(43) Pub. Date: Mar. 15, 2018

(54) METHODS FOR TREATING ALZHEIMER'S DISEASE

(71) Applicant: Reviva Pharmaceuticals, Inc., Santa Clara, CA (US)

(72) Inventors: Laxminarayan BHAT, Santa Clara, CA (US); Marc Cantillon, Santa Clara, CA (US); Seema Rani Bhat, Santa Clara, CA (US)

(21) Appl. No.: 15/643,718

(22) Filed: Jul. 7, 2017

Related U.S. Application Data
(63) Continuation of application No. PCT/US2016/013061, filed on Jan. 12, 2016.
(60) Provisional application No. 62/102,513, filed on Jan. 12, 2015.

(51) Int. Cl. A61K 31/538; A61K 9/00

(52) U.S. Cl. CPC A61K 31/538; A61K 9/0053

(57) ABSTRACT

The present invention provides a method of using arylpiperazine derivatives for treating behavioral and psychological symptoms in Alzheimer's disease patients. The method comprises a step of administering to a patient in need thereof an effective amount of a compound of Formula 1, which is an arylpiperazine derivative.

This application is a continuation of PCT/US2016/013061, filed Jan. 12, 2016; which claims the priority of U.S. Provisional Application No. 62/102,513, filed Jan. 12, 2015. The contents of the above-identified applications are incorporated herein by reference in their entireties.

### TECHNICAL FIELD

The present invention relates to methods of utilizing arylpiperazine derivatives for treating behavioral and psychological symptoms in Alzheimer's disease.

### BACKGROUND

Dementia is a clinical syndrome with features of neurocognitive decline. Subtypes of dementia include Alzheimer's, frontotemporal, Lewy body disease, and vascular type. Dementia is associated with a variety of neuropsychiatric symptoms that may include agitation, psychosis, depression, and apathy. These symptoms can lead to danger to self or others and are the main source for caregiver burnout. Treatment of these symptoms consists of nonpharmacological and pharmacological interventions. However, there are no Food and Drug Administration-approved medications for the treatment of behavioral and psychological symptoms of dementia (BPSD). Pharmacological interventions for BPSD are used off-label, most commonly antipsychotics (commonest), anticonvulsants or anxiolytics. The second generation antipsychotics (SGA) are widely used for treating BPSD. However, a recent meta-analysis based placebo controlled trials of elderly patients with BPSD revealed only modest effects for three SGA, that is, risperdone, olanzapine and aripiprazole (Maher et al., 2011). In addition, the second generation antipsychotics are reported to produce adverse effects including extrapyramidal symptoms (EPS), cardiovascular and metabolic side effects (Nobili et al., 2009; Schlze et al., 2013). Moreover the second generation antipsychotics may worsen cognitive functioning, which can be a substantial drawback in the case of elderly patients who already suffer from cognitive deficits (Jeste et al., 2008, Vigen et al., 2011). It has been reported that psychotic symptoms in schizophrenia patients are different than often observed in dementia patients (Jeste and Finkel et al., 2000). The distinct nature of psychotic symptoms in dementia suggests that different neurobiological mechanisms are involved. In particular, serotonergic systems besides because hallucinations in dementia are similar to those caused by serotonergic agonists such as mescaline or lysergic acid (Marsh, 1979). Currently available antipsychotics were selected primarily for their capacity to inhibit the effects of dopaminomimetics, and therefore, may potentially provide suboptimal therapeutic efficacy in the treatment of BPSD. In particular, they were not optimized to provide treat mood deficits or to avoid accentuating cognitive deficits in elderly patients.

Thus, there is a need for more effective therapies for treating behavioral and psychological symptoms in Alzheimer's disease.

### DETAILED DESCRIPTION OF THE INVENTION

### Definitions

“Alkyl” or “alkanyl” refers to a saturated, branched or straight-chain or cyclic monovalent hydrocarbon radical derived by the removal of one hydrogen atom from a single carbon atom of a parent alkane. Typical alkyl groups include, but are not limited to methyl; ethyl; propyls such as propan-1-yl, propan-2yl, cyclopropan-1-yl; butyls such as butan-1-yl, butan-2-yl, 2-methyl-propan-1-yl, 2-methyl-propan-2-yl, cyclobutan-1-yl and the like. Preferably, an alkyl group comprises from 1-20 carbon atoms, more preferably, from 1 to 10, or 1 to 6, or 1-4 carbon atoms.

“Alkenyl” refers to an unsaturated branched, straight-chain or cyclic alkyl radical having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl, cycloprop-2-en-1-yl; butenyls such as but-1-en-1-yl, but-1-en-2-yl, 2-methy-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien 1-yl, etc.; and the like.

“Alkynyl” refers to an unsaturated branched, straight-chain or cyclic alkyl radical having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn3-yl, but-3-yn-1-yl, etc.; and the like.

“Acyl” refers to a radical —C(O)R, where R is hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to formyl, acetyl, cyclohexylcarbonyl, cyclohexylmethylcarbonyl, benzoyl, benzylcarbonyl and the like.

“Acyloxyalkyloxycarbonyl” refers to a radical —C(O)OCR′R″OC(O)R′″, where R′, R″, and R′″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but not limited to —C(O)OCH₂OC(O)CH₃, —C(O)OCH₂OC(O)CH₂CH₃, —C(O)OCH(CH₃)OC(O)CH₂CH₃, —C(O)OCH(CH₃)OC(O)C₆H₅ and the like.

“Acylalkyloxycarbonyl” refers to a radical —C(O)OCR′R″C(O)R′″, where R′, R″, and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but not limited to —C(O)OCH₂C(O)CH₃, —C(O)OCH₂C(O)CH₂CH₃, —C(O)OCH(CH₃)C(O)CH₂CH₃, —C(O)OCH(CH₃)C(O)C₆H₅ and the like.

“Acyloxyalkyloxycarbonylamino” refers to a radical —NRC(O)OCR′R″OC(O)R′″, where R, R′, R″, and R′″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but not limited to —NHC(O)OCH₂OC(O)CH₃, —NHC(O)OCH₂OC(O)CH₂CH₃, —NHC(O)OCH(CH₃)OC(O)CH₂CH₃, —NHC(O)OCH(CH₃)OC(O)C₆H₅ and the like.

“Acylalkyloxycarbonylamino” refers to a radical —NRC(O)OCR′R″C(O)R′″, where R, R′, R″, and R′″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but not limited to —NHC(O)OCH₂C(O)CH₃, —NHC(O)OCH₂C(O)CH₂CH₃, —NHC(O)OCH(CH₃)C(O)CH₂CH₃, —NHC(O)OCH(CH₃)C(O)C₆H₅ and the like.

“Acylamino” refers to “amide” as defined herein.

“Alkylamino” means a radical —NHR where R represents an alkyl, or cycloalkyl group as defined herein that may be optionally substituted by one or more substituents as defined herein. Representative examples include, but are not limited to, methylamino, ethylamino, 1-methylethylamino, cyclohexylamino and the like.

“Alkoxy” refers to a radical —OR where R represents an alkyl, or cycloalkyl group as defined herein that may be optionally substituted by one or more substituents as defined herein. Representative examples include, but are not limited to methoxy, ethoxy, propoxy, butoxy, cyclohexyloxy and the like.

“Alkoxycarbonyl” refers to a radical —C(O)-alkoxy where alkoxy is as defined herein.

“Alkoxycarbonylalkoxy” refers to a radical —OCR′R″C(O)-alkoxy where alkoxy is as defined herein. Similarly, where R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to —OCH₂C(O)OCH₃, —OCH₂C(O)OCH₂CH₃, —OCH(CH₃)C(O)OCH₂CH₃, —OCH(C₆H₅)C(O)OCH₂CH₃, —OCH(CH₂C₆H₅)C(O)OCH₂CH₃, —OC(CH₃)(CH₃)C(O)OCH₂CH₃, and the like.

“Alkoxycarbonylalkylamino” refers to a radical —NRCR′R″C(O)-alkoxy where alkoxy is as defined herein. Similarly, where R, R′, R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to —NHCH₂C(O)OCH₃, —N(CH₃)CH₂C(O)OCH₂CH₃, —NHCH(CH₃)C(O)OCH₂CH₃, —NHCH(C₆H₅)C(O)OCH₂CH₃, —NHCH(CH₂C₆H₅)C(O)OCH₂CH₃, —NHC(CH₃)(XCH₃)C(O)OCH₂CH₃, and the like.

“Alkylsulfonyl” refers to a radical-S(O)₂R where R is an alkyl, or cycloalkyl group as defined herein that may be optionally substituted by one or more substituents as defined herein. Representative examples include, but are not limited to, methylsulfonyl, ethylsulfonyl, propylsulfonyl, butylsulfonyl, and the like.

“Alkylsulfinyl” refers to a radical —S(O)R where R is an alkyl, or cycloalkyl group as defined herein that may be optionally substituted by one or more substituents as defined herein. Representative examples include, but are not limited to, methylsulfinyl, ethylsulfinyl, propylsulfinyl, butylsulfinyl, and the like.

“Alkylthio” refers to a radical —SR where R is an alkyl or cycloalkyl group as defined/herein that may be optionally substituted by one or more substituents as defined herein. Representative examples include, but are not limited to methylthio, ethylthio, propylthio, butylthio, and the like.

“Amide” or “acylamino” refers to a radical —NR′C(O)R″, where R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to, formylamino acetylamino, cyclohexylcarbonylamino, cyclohexylmethylcarbonyl-amino, benzoylamino, benzylcarbonylamino and the like.

“Aryl” refers to a monovalent aromatic hydrocarbon radical derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorine, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleidene, pyrene, pyranthrene, rubicene, triphenylene, trinaphthalene, and the like. Preferable, an aryl group comprises from 6 to 20 carbon atoms, more preferably, between 6 to 12 carbon atoms.

“Arylalkyl” refers to an acyclic alkyl in which one of the hydrogen atoms bonded to a carbon atom, typically a terminal or sp³ carbon atom, is replaced with an aryl group. Typically arylalkyl groups include, but not limited to, benzyl, 2-phenylethan-1-yl, naphthylmethyl, 2-naphthylethan-1-yl, naphthobenzyl, 2-naphthophenylethan-1-yl and the like. Preferably, an arylalkyl group is (C₆-C₃₀)arylalkyl, e.g., the alkyl moiety of the arylalkyl group is (C₁-C₁₀) and the aryl moiety is (C₆-C₂₀), more preferably, an arylalkyl group is (C₆-C₂₀) arylalkyl, e.g., the alkyl moiety of the arylalkyl group is (C₁-C₈) and the aryl moiety is (C₆-C₁₂).

“Arylalkoxy” refers to an —O-arylalkyl radical where arylalkyl is as defined herein that may be optionally substituted by one or more substituents as defined herein.

“Arylalkoxycarbonylalkoxy” refers to a radical —OCR′R″C(O)-arylalkoxy where arylalkoxy is as defined herein. Similarly, where R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to —OCH₂C(O)OCH₂C₆H₅, —OCH(CH₃)C(O)OCH₂C₆H₅, —OCH(CH₅)C(O)OCH₂C₆H₅, —OCH(CH₂C₆H₅)C(O)OCH₂C₆H₅, —OC(CH₃)(CH₃)C(O)OCH₂C₆H₅, and the like.

“Arylalkoxycarbonylalkylamino” refers to a radical —NRCR′R″C(O)-arylalkoxy where arylalkoxy is as defined herein. Similarly, where R, R′, R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to —NHCH₂C(O)OCH₂C₆H₅, —N(CH₃)CH₂C(O)OCH₂C₆H₅, —NHCH(CH₃)C(O)OCH₂C₆H₅, —NHCH(C₆H₅)C(O)OCH₂C₆H₅, —NHCH(CH₂C₆H₅)C(O)OCH₂C₆H₅, —NHC(CH₃)(CH₃)C(O)OCH₂C₆H₅, and the like.

“Aryloxycarbonyl” refers to radical —C(O)—O-aryl where aryl is defined herein that may be optionally substituted by one or more substituents as defined herein.

“Aryloxycarbonylalkoxy” refers to a radical —OCR′R″C(O)-aryloxy where aryloxy is as defined herein. Similarly, where R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to —OCH₂C(O)OC₆H₅, —OCH(CH₃)C(O)OC₆H₅, —OCH(C₆H₅)C(O)OC₆H₅, —OCH(CH₂C₆H₅)C(O)OC₆H₅, —OC(CH₃)(CH₃)C(O)OC₆H₅, and the like.

“Aryloxycarbonylalkylamino” refers to a radical —NRCR′R″C(O)-aryloxy where aryloxy is as defined herein. Similarly, where R, R′, R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to —NHCH₂C(O)OC₆H₅, —N(CH₃)CH₂C(O)OC₆H₅, —NHCH(CH₃)C(O)OC₆H₅, —NHCH(C₆H₅)C(O)OC₆H₅, —NHCH(CH₂C₆H₅)C(O)OC₆H₅, —NHC(CH₃)(XCH₃)C(O)OC₆H₅, and the like.

“Carbamoyl” refers to the radical —C(O)NRR where each R group is independently, hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein.

“Carbamate” refers to a radical —NR′C(O)OR″, where R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to, methylcarbamate (—NHC(O)OCH₃), ethylcarbamate (—NHC(O)OCH₂CH₃), benzylcarbamate (—NHC(O)OCH₂C₆H₅), and the like.

“Carbonate” refers to a radical —OC(O)OR, where R is alkyl, cycloalkyl, cycloheteroalkyl, aryl, arylalkyl, heteroalkyl, heteroaryl, heteroarylalkyl, as defined herein that may be optionally substituted by one or more substituents as defined herein. Representative examples include, but are not limited to, methyl carbonate (—C(O)OCH₃), cyclohexyl carbonate (—C(O)OC₆H₁), phenyl carbonate (—C(O)OC₆H₅), benzyl carbonate (—C(O)OCH₂C₆H₅), and the like.

“Cycloalkyl” refers to a substituted or unsubstituted cyclic alkyl radical. Typical cycloalkyl groups include, but are not limited to, groups derived from cyclopropane, cyclobutane, cyclopentane, cyclohexane, and the like. In a preferred embodiment, the cycloalkyl group is (C₃-C₁₀) cycloalkyl, more preferably (C₃-C₇) cycloalkyl.

“Cycloheteroalkyl” refers to a saturated or unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, imidazolidine, morpholine, piperazine, piperidine, pyrazolidine, pyrrolidine, quinuclidine, and the like.

“Cycloheteroalkoxycarbonyl” refers to a radical —C(O)—OR where R is cycloheteroalkyl as defined herein that may be optionally substituted by one or more substituents as defined herein.

“Dialkylamino” means a radical —NRR′ where R and R′ independently represent an alkyl or cycloalkyl group as defined herein that may be optionally substituted by one or more substituents as defined herein. Representative examples include, but are not limited to dimethylamino, methylethylamino, di-(1-methylethyl)amino, (cyclohexyl)(methyl)amino, (cyclohexyl)(ethyl)amino, (cyclohexyl)(propyl)amino, and the like.

“Ester” refers to a radical —C(O)OR, where R is alkyl, substituted alkyl, cycloalkyl, substituted cycloalkyl, cycloheteroalkyl, substituted cycloheteroalkyl, aryl, substituted aryl, arylalkyl, substituted arylalkyl, heteroalkyl, substituted heteroalkyl, heteroaryl, substituted heteroaryl, heteroarylalkyl, substituted heteroarylalkyl as defined herein that may be optionally substituted by one or more substituents as defined herein. Representative examples include, but are not limited to, methyl ester (—C(O)OCH₃), cyclohexyl ester (—C(O)OC₆H₁), phenyl ester (—C(O)OC₆H₅), benzyl ester (—C(O)OCH₂C₆H₅), and the like.

“Ether” refers to a radical —OR, where R is alkyl, cycloalkyl, cycloheteroalkyl, aryl, arylalkyl, heteroalkyl, heteroaryl, heteroarylalkyl, as defined herein that may be optionally substituted by one or more substituents as defined herein.

“Halogen” means fluoro, chloro, bromo, or iodo.

“Heteroaryl” refers to a monovalent heteroaromatic radical derived by the removal of one hydrogen atom from a single atom of a parent heteroaromatic ring system. Typical heteroaryl groups include, but are not limited to, groups derived from acridine, arsindole, carbazole, carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. Preferably, the heteroaryl group is between 5-20 membered heteroaryl, with 5-10 membered heteroaryl being particularly preferred. Preferred heteroaryl groups are those derived from thiophene, pyrrole, benzothiophene, benzofuran, indole, pyridine, quinoline, imidazole, oxazole and pyrazine.

“Heteroaryloxycarbonyl” refers to a radical —C(O—OR where R is heteroaryl as defined that may be optionally substituted by one or more substituents as defined herein.

“Heteroarylalkyl” refers to an acyclic alkyl radical in which one of the hydrogen atoms bonded to a carbon atom, typically a terminal or sp3 carbon atom, is replaced with a heteroaryl group. Preferably, the heteroarylalkyl radical is a 6-30 carbon membered heteroarylalkyl, e.g., the alkyl moiety of the heteroarylalkyl is 1-10 membered and the heteroaryl moiety is a 5-20 membered heteroaryl, more preferably, a 6-20 membered heteroarylalkyl, e.g., the alkyl moiety of the heteroarylalkyl is 1-8 membered and the heteroaryl moiety is a 5-12 membered heteroaryl.

“Oxo” means the divalent radical ═O.

“Pharmaceutically acceptable” means approved or approvable by a regulatory agency of the Federal or state government or listed in the U.S. Pharmacopoeia or other generally recognized pharmacopoeia for use in animals, and more particularly in humans.

“Pharmaceutically acceptable salt” refers to a salt of a compound of the invention, which is pharmaceutically acceptable and possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentane propionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2,2,2]-oct-2-ene-1-carboxylic acid, glucoheptonic acid, 3-phenylpropionic acid, trimethylacetic acid, tertiary butylacetic acid, laurylsulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Pharmaceutically acceptable vehicle” refers to a diluent, adjuvant, excipient or carrier with which a compound of the invention is administered.

“Phosphate” refers to a radical —OP(O)(OR′)(OR″), where R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein.

“Phosphonate” refers to a radical —P(O)(OR′)(OR″), where R′ and R″ are each independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein.

“Preventing” or “Prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease).

“Racemate” refers to an equimolar mixture of enantiomers of a chiral molecule.

“Substituted” refers to a group in which one or more hydrogen atoms are each independently replaced with the same or different substituents(s). Typical substituents include, but are not limited to, —X, —R⁵⁴, —O⁻, ═O, —OR⁵⁴, —SR⁵⁴, —S, ═S, —NR⁵⁴R⁵⁵, ═NR⁵⁴, —CX₃, —CF₃, —CN, —OCN, —SCN, —NO, —NO₂, ═N₂, —N₃, —S(O)₂O⁻, —S(O)₂OH, —S(O)₂OR⁵⁴, —OS(O)₂O³¹, —OS(O)₂R⁵⁴, —P(O)(O—)₂, —P(O)(OR¹⁴)(O³¹), —OP(O)(OR⁵⁴)(OR⁵⁵), —C(O)R⁵⁴, —C(S)R⁵⁴, —C(O)OR⁵⁴, —C(O)NR⁵⁴R⁵⁵, —C(O)O⁻, —C(S)OR⁵⁴, —NR⁵⁶C(O)NR⁵⁴R⁵⁵, —NR⁵⁶C(S)NR⁵⁴R⁵⁵, —NR⁵⁷C(NR⁵⁶)NR⁵⁴R⁵⁵, and —C(NR⁵⁶)NR⁵⁴R⁵⁵, where each X is independently a halogen; each R⁵⁴, R⁵⁵, R⁵⁶ and R⁵⁷ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, substituted heteroarylalkyl, —NR⁵⁸R⁵⁹, —C(O)R⁵⁸ or —S(O)₂R⁵⁸ or optionally R⁵⁸ and R⁵⁹ together with the atom to which they are both attached form a cycloheteroalkyl or substituted cycloheteroalkyl ring; and R⁵⁸ and R⁵⁹ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, substituted heteroarylalkyl.

“Sulfate” refers to a radical —OS(O)(O)OR, where R is hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein.

“Sulfonamide” refers to a radical —S(O)(O)NR′R″, where R′ and R″ are independently hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein or optionally R′ and R″ together with the atom to which they are both attached form a cycloheteroalkyl or substituted cycloheteroalkyl ring. Representative examples include but not limited to azetidinyl, pyrrolidinyl, piperidinyl, morpholinyl, 4-(NR′″)-piperazinyl or imidazolyl group wherein said group may be optionally substituted by one or more substituents as defined herein. R′″ hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein.

“Sulfonate” refers to a radical-S(O)(O)OR, where R is hydrogen, alkyl, cycloalkyl, cycloheteroalkyl, aryl, arylalkyl, heteroalkyl, heteroaryl, heteroarylalkyl, as defined herein that may be optionally substituted by one or more substituents as defined herein.

“Thio” means the radical —SH.

“Thioether” refers to a radical —SR, where R is alkyl, cycloalkyl, cycloheteroalkyl, aryl, arylalkyl, heteroalkyl, heteroaryl, heteroarylalkyl, as defined herein that may be optionally substituted by one or more substituents as defined herein.

“Treating” or “Treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the patient. In yet another embodiment, “treating” or “treatment” refers to inhibiting the disease or disorder, either physically (e.g., stabilization of a discernible symptom), physiologically, (e.g., stabilization of a physical parameter), or both.

“Therapeutically effective amount” means the amount of a compound that, when administered to a patient for treating a disease, is sufficient to effect such treatment for the disease. The “therapeutically effective amount” will vary depending on the compound, the disease and is severity and the age, weight, etc., of the patient to be treated, and can be determined by one of skill in the art without undue experimentation.

The present invention is directed to a method for treating behavioral and psychological symptoms in Alzheimer's disease.

### Compounds Useful in the Invention

Compounds of Formula (I) are useful for the present invention:

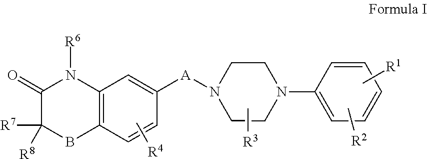

wherein:
A is —(CH₂)ₙ—, —O—(CH₂)ₙ—, —S—(CH₂)ₙ—, —S(O)(O)—(CH₂)ₙ—, —NH—(CH₂)ₙ—, —CH₂—O—(CH₂)ₙ—, —(CH₂)ₙ—O—CH₂—CH₂—, —CH₂—S—(CH₂)ₙ—, —(CH₂)ₙ—S—CH₂—CH₂—, —CH₂—S(O)(O)—(CH₂)ₙ—, —(CH₂)ₙ—S(O)(O)—CH₂—CH₂—, —O—C(O)—(CH₂)ₙ—, —S—C(O)—(CH₂)ₙ—, —NH—C(O)—(CH₂)ₙ—, —CH₂—C(O)—O—(CH₂)ₙ—, —CH₂—C(O)—NH—(CH₂)ₙ—, —CH₂—C(O)—S—(CH₂)ₙ—, —(CH₂)ₙ—C(O)—O—CH₂—CH₂—, —(CH₂)ₙ—C(O)—NH—CH₂—CH₂—, —(CH₂)ₙ—C(O)—S—CH₂—CH₂—, —CH₂—O—C(O)—(CH₂)ₙ—, —CH₂—NH—C(O)—(CH₂)ₙ—, —CH₂—S—C(O)—(CH₂)ₙ—, —(CH₂)ₙ—O—C(O)—CH₂—CH₂—, (CH₂)ₙ—NH—C(O)—CH₂—CH₂—, or (CH₂)ₙ—S—C(O)—CH₂—CH₂—, wherein n is an integer from 1 to 7, preferably n is 2 to 5, for example n is 4;
B is O, S, S(O)(O), or NR⁵; and
each of R¹, R², R³, R⁴, R⁵, R⁶, R⁷, and R⁸ is independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl, substituted heteroarylalkyl, acylalkyloxycarbonyl, acyloxyalkyloxycarbonyl, acylalkyloxycarbonylamino, acyloxyalkyloxycarbonylamino, alkoxy, alkoxycarbonyl, alkoxycarbonylalkoxy, alkoxycarbonyllalkylamino, alkylsulfinyl, alkylsulfonyl, alkylthio, amino, alkylamino, arylalkylamino, dialkylamino, arylalkoxy, arylalkoxycarbonylalkoxy, arylalkoxycarbonylalkylamino, aryloxycarbonyl, aryloxycarbonylalkoxy, aryloxycarbonylalkylamino, carboxy, carbamoyl, carbamate, carbonate, cyano, halo, heteroaryloxycarbonyl, hydroxy, phosphate, phosphonate, sulfate, sulfonate, or sulfonamide, wherein R¹, R², R³, R⁴, R⁵, R⁶, R⁷ and R⁸ and A may optionally be substituted with isotopes that include, but not limited to ²H (deuterium), ³H (tritium), ¹³C, ³⁶Cl, ¹⁸F, ¹⁵N, ¹⁷O, ¹⁸O, ³¹P, ³²P, and ³⁵S; with ²H (deuterium) being preferred;
or a pharmaceutically acceptable salt, racemate or diastereomeric mixtures thereof.

In one aspect of the invention, A is —(CH₂)ₙ—.

In another aspect of the invention, A is —O—(CH₂)ₙ—, —S—(CH₂)ₙ—, —CH₂—O—(CH₂)ₙ—, —(CH₂)ₙ—O—CH₂—CH₂—, —CH₂—S—(CH₂)ₙ—, or —(CH₂)ₙ—S—CH₂—CH₂—; with A being —O—(CH₂)ₙ—, for example, —O—(CH₂)₄—, preferred.

In another aspect of the invention, A is —NH—C(O)—(CH₂)ₙ—, —CH₂—NH—C(O)—(CH₂)ₙ—, —CH₂—C(O)—NH—(CH₂)ₙ— or —(CH₂)ₙ—C(O)—NH—CH₂—CH₂—.

In another aspect of the invention, B is O.

In another aspect of the invention, R³, R⁴, R⁶, R⁶, and R⁸ are H.

In another aspect of the invention, each of R¹ and R² is independently H, halogen (e.g., chloro), haloalkyl, or alkoxy (e.g., methoxy or ethoxy); preferably halogen or alkoxy.

In a preferred embodiment, A is —O—(CH₂)ₙ—, n=2-5; B is O; R³, R⁴, R⁶, R⁶, and R⁸ are H; and R¹ and R² is independently H, halogen, haloalkyl, or alkoxy.

Preferred compounds of Formula I include, for example,

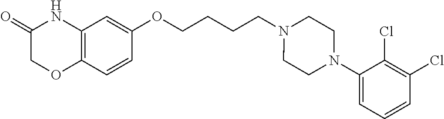 [SMILES: O=C1COc2ccc(OCCCCN3CCN(c4cccc(Cl)c4Cl)CC3)cc2N1]

6-(4-(4-(2,3-dichlorophenyl)piperazin-1-yl)butoxy)-2H-benzo[b][1,4]oxazin-3(-4H)-one, and its hydrochloride salt (Compound A); and

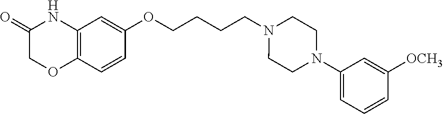 [SMILES: COc1cccc(N2CCN(CCCCOc3ccc4c(c3)NC(=O)CO4)CC2)c1]

6-(4-(4-(2-methoxyphenyl)piperazin-1-yl)butoxy)-2H-benzo[b][1,4]oxazin-3(-4H)-one, and its hydrochloride salt (Compound B).

The compounds useful for the present invention further pertain to enantiomerically isolated compounds of Formula I. The isolated enantiomeric forms of the compounds of Formula I are substantially free from one another (i.e., in enantiomeric excess). In other words, the “R” forms of the compounds are substantially free from the “S” forms of the compounds and are, thus, in enantiomeric excess of the “S” forms. Conversely, “S” forms of the compounds are substantially free of “R” forms of the compounds and are, thus, in enantiomeric excess of the “R” forms. In one embodiment of the invention, the isolated enantiomeric compounds are at least about in 80% enantiomeric excess. Thus, for example, the compounds are at least about 90% enantiomeric excess, preferably at least about 95% enantiomeric excess, more preferably at least about 97% enantiomeric excess, or even more preferably, at least 99% or greater than 99% enantiomeric excess.

Formula I compounds can be synthesized according U.S. Pat. No. 8,188,076, which is incorporated herewith in its entirety.

### Method of Treating Behavioral and Psychological Symptoms in Alzheimer's Disease Patients

The present invention is directed to a method for treating behavioral and psychological symptoms in Alzheimer's disease patients. The method comprises the step of administering an effective amount of a compound of Formula I to a patient in need thereof.

The present invention is effective for treating behavioral and psychological symptoms in patients having Alzheimer's disease. Behavorial and psychological symptoms of dementia are caused by imbalance of dopamine-serotonin systems in the brain. Compounds of Formula I have potent binding affinity at the serotonin 5-HT2A receptor (compound B, Ki=2.5 nM, see Example 1) and 5-HT2B receptor (compound B, Ki=0.19 nM, see Example 1). In addition, compounds of Formula I exhibit partial agonist activities for the key subtypes of dopamine (D1, D2, D3 and D4) and serotonin (5-HT1A), and antagonist activity at the serotonin 5-HT6 and 5-HT7 receptors. Compounds of Formula I are potent dopamine and serotonin system modulators due to their selectivity, potent binding affinities, and especially partial agonist activities for key dopamine and serotonin receptors. Due to the unique interaction of compounds of Formula I to various dopamine and serotonin receptors, the inventors have discovered that Formula I compounds are effective for treating behavioral and psychological symptoms in Alzheimer's disease.

In one embodiment, Formula I compounds treats memory impairment in patients having Alzheimer's disease

In one embodiment, Formula I compounds treats cognitive impairment in Alzheimer's disease

In one embodiment, Formula I compounds treats agitation in Alzheimer's disease.

In one embodiment, Formula I compounds treats mood swing in Alzheimer's disease.

In one embodiment, Formula I compounds treats psychosis in Alzheimer's disease.

In one embodiment, Formula I compounds treats depression in Alzheimer's disease.

Formula I compounds are effective in reducing the behavioral and psychological symptoms of dementia (BPSD), particularly the signs and symptoms of agitation in dementia of Alzheimer's type. The treatment in general improves the primary outcomes such as Neuropsychiatric Inventory (NPI), function (Bristol Activities of Daily Living Scale, BADLS) and agitation (Cohen-Mansfield Agitation Inventory, CMAI). The treatment may also improve secondary outcomes such as Mini-Mental State Examination (MMSE), general functioning, caregiver burden and mortality.

When used to treat behavioral and psychological symptoms in patients having Alzheimer's disease, one or more compound of Formula I can be administered alone, or in combination with other agents, to a patient. The patient may be an animal, preferably a mammal, and more preferably a human.

Formula I compounds are preferably administered orally. Formula I compounds may also be administered by any other convenient route, for example, by infusion or bolus injection, by absorption through epithelial or mucocutaneous linings (e.g., oral mucosa, rectal and intestinal mucosa, etc.). Administration can be systemic or local. Various delivery systems are known, (e.g., encapsulation in liposomes, microparticles, microcapsules, capsules, etc.) that can be used to administer a compound and/or composition of the invention. Methods of administration include, but are not limited to, intradermal, intramuscular, intraperitoneal, intravenous, subcutaneous, intranasal, epidural, oral, sublingual, intranasal, intracerebral, intravabinal, transdermal, rectally, by inhalation, or topically, particularly to the ears, nose, eyes or skin. Transdermal administration may be preferred for young children.

Formula I compounds can be delivered via sustained release systems, preferably oral sustained release systems. In one embodiment, a pump may be used (see, Langer, supra; Sefton, 1987, CRC Crit. Ref Biomed. Eng. 14:201; Saudek et al., 1989, N. Engl. J. Med. 321:574).

In one embodiment, polymeric materials can be used (see “Medical Applications of Controlled Release,” Langer and Wise (eds.), Wiley, New York (1984); Ranger and Peppas, 1983, J. Macromol. Sci. Rev. Macromol Chem. 23:61; see also Levy et al., 1985, Science 228:190; During et al., 1989, Ann. Neurol. 25:351; Howard et al, 1989, J. Neurosurg. 71:105). In a preferred embodiment, polymeric materials are used for oral sustained release delivery. Preferred polymers include sodium carboxymethylcellulose, hydroxypropylcellulose, hydroxypropylmethylcellulose and hydroxyethylcellulose (most preferred, hydroxypropylmethylcellulose). Other preferred cellulose ethers have been described in the art (Bamba et al., Int. J. Pharm., 1979, 2, 307).

In one embodiment, enteric-coated preparations can be used for oral sustained release administration. Preferred coating materials include polymers with a pH-dependent solubility (i.e., pH-controlled release), polymers with a slow or pH-dependent rate of swelling, dissolution or erosion (i.e., time controlled release), polymers that are degraded by enzymes (i.e., enzyme controlled release) and polymers that form firm layers that are destroyed by an increase in pressure (i.e., pressure-controlled release).

In still another embodiment, osmotic delivery systems are used for oral sustained release administration (Verma et al., Drug Dev. Ind. Pharm., 2000, 26:695-708). In a preferred embodiment, OROS® osmotic delivery systems are used for oral sustained release delivery devices (See for example, Theeuwes et al., U.S. Pat. No. 3,845,770; and Theeuwes et al, U.S. Pat. No. 3,916,899).

In yet another embodiment, a controlled-release system can be placed in proximity of the target of the compounds and/or composition of the invention, thus requiring only a fraction of the systemic dose (See, e.g., Goodson, in “Medical Applications of Controlled Release,” supra, vol. 2, pp. 115-138 (1984)). Other controlled-release systems discussed in Langer, 1990, Science 249:1527-1533 may also be used.

Formula I compounds may be cleaved either chemically and/or enzymatically. One or more enzymes present in the stomach, intestinal lumen, intestinal tissue, blood, liver, brain or any other suitable tissue of a mammal may enzymatically cleave the compounds and/or compositions of the invention.

### Pharmaceutical Formulation of the Invention

The present invention is directed to a pharmaceutical formulation for treating Alzheimer disease. The pharmaceutical formulation contains a therapeutically effective amount of one or more compounds of Formula I, preferably in purified form, together with a suitable amount of a pharmaceutically acceptable vehicle. When administered to a patient, the pharmaceutical formulation is preferably sterile. Water is a preferred vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present agents, or pH buffering agents. In addition, auxiliary, stabilizing, thickening, lubricating and coloring agents may be used.

Pharmaceutical compositions comprising a compound of the invention may be manufactured by means of conventional mixing, dissolving, granulating, levigating, and emulsifying, encapsulating, entrapping or lyophilizing process. Pharmaceutical compositions may be formulated in conventional manner using one or more physiologically acceptable carriers, diluents, excipients or auxiliaries, which facilitate processing of compounds of the invention into preparations which can be used pharmaceutically. Proper formulation is dependent upon the route of administration chosen.

The present compositions can take the form of solutions, suspensions, emulsion, tablets, pills, pellets, and capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., Grosswald et al., U.S. Pat. No. 5,698,155). Other examples of suitable pharmaceutical vehicles have been described in the art (see Remington's Pharmaceutical Sciences, Philadelphia College of Pharmacy and Science, 17^{th} Edition, 1985). Preferred compositions of the invention are formulated for oral delivery, particularly for oral sustained release administration.

Compositions for oral delivery may be in the form of tablets, lozenges, aqueous or oily suspensions, granules, powders, emulsions, capsules, syrups or elixirs, for example. Orally administered compositions may contain one or more optionally agents, for example, sweetening agents such as fructose, aspartame or saccharin; flavoring agents such as peppermint, oil of wintergreen, or cherry coloring agents and preserving agents to provide a pharmaceutically palatable preparation. Moreover, where in tablet or pill form, the compositions may be coated to delay disintegration and absorption in the gastrointestinal tract, thereby providing a sustained action over an extended period of time. Selectively permeable membranes surrounding an osmotically active driving compound are also suitable for orally administered compounds of the invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

For oral liquid preparations such as, for example, suspensions, elixirs and solutions, suitable carriers, excipients or diluents include water, saline, alkyleneglycols (e.g., propylene glycol), polyalkylene glycols (e.g., polyethylene glycol) oils, alcohols, slightly acidic buffers between pH 4 and pH 6 (e.g., acetate, citrate, ascorbate at between about mM to about 50 mM) etc. Additionally, flavoring agents, preservatives, coloring agents, bile salts, acylcarnitines and the like may be added.

Compositions for administration via other routes may also be contemplated. For buccal administration, the compositions may take the form of tablets, lozenges, etc. formulated in conventional manner. Liquid drug formulations suitable for use with nebulizers and liquid spray devices and EHD aerosol devices will typically include a compound of the invention with a pharmaceutically acceptable vehicle. Preferably, the pharmaceutically acceptable vehicle is a liquid such as alcohol, water, polyethylene glycol or a perfluorocarbon. Optionally, another material may be added to alter the aerosol properties of the solution or suspension of compounds of the invention. Preferably, this material is liquid such as alcohol, glycol, polyglycol or fatty acid. Other methods of formulating liquid drug solutions or suspension suitable for use in aerosol devices are known to those of skill in the art (see, e.g., Biesalski, U.S. Pat. No. 5,112,598; Biesalski, U.S. Pat. No. 5,556,611). A compound of the invention may also be formulated in rectal or vaginal compositions such as suppositories or retention enemas, e.g., containing conventional suppository bases such as cocoa, butter or other glycerides. In addition to the formulations described previously, a compound of the invention may also be formulated as depot preparation. Such long acting formulations may be administered by implantation (for example, subcutaneously or intramuscularly) or by intramuscular injection. Thus, for example, a compound of the invention may be formulated with suitable polymeric or hydrophobic materials (for example, as an emulsion in an acceptable oil) or ion exchange resins, or as sparingly soluble derivatives, for example, as a sparingly soluble salt.

### Dosage for the Treatment

The amount of Formula I compound administered is dependent on, among other factors, the subject being treated, and the weight of the subject, the severity of the affliction, the manner of administration and the judgment of the prescribing physician. For example, the dosage may be delivered in a pharmaceutical composition by a single administration, by multiple applications or controlled release. In one embodiment, the compounds of the invention are delivered by oral sustained release administration. In one embodiment, the compounds of the invention are administered twice per day, and preferably, once per day. Dosing may be repeated intermittently, may be provided alone or in combination with other drugs, and may continue as long as required for effective treatment of the disease state or disorder.

The compounds of Formula I may be administered in the range 0.1 mg to 500 mg, preferably 1 mg to 100 mg per day, such as 5 mg, 10 mg, 15 mg, 20 mg, 25 mg, 35 mg or 50 mg per day, and preferably 10 mg per day.

### Combination Therapy

In certain embodiments of the present invention, the compounds of the invention can be used in combination therapy with at least one other therapeutic agent. Formula I compounds and the therapeutic agent can act additively or synergistically. In one embodiment, Formula I compound is administered concurrently with the administration of another therapeutic agent, which can be part of the same composition of Formula I compound. In another embodiment, a composition comprising a compound of the invention is administered prior or subsequent to administration of another therapeutic agent.

The invention is further illustrated by the following examples.

### EXAMPLES

### Example 1. In Vitro Pharmacology Results

Two arylpiperazine derivatives of Formula (I) were tested in the in vitro pharmacological assays to evaluate their activities for dopamine—D₁, D_{2L}, D_{2S}, D₃, D_{4.4}; serotonin—5-HT_{1A}, 5-HT_{2A}, 5-HT_{2B}, 5-HT₆, 5-HT₇; serotonin transporter (SERT); and nicotinic acetylcholine alpha4beta2 (nACh-α₄β₂)serotonin. The radioligand binding assays were carried out at six to 10 different concentrations and the test concentrations were 0.1 nM, 0.3 nM, 1 nM, 10 nm, 30 nM, 100 nM, 300 nM, 1000 nM, 10000 nM. The in vitro assay protocols and literature references are described herein.

### Dopamine, Dᵣ Radioligand Binding Assay

### Materials and Methods:

Receptor Source: Human recombinant D₁ expressed CHO cells

### Radioligand: [3H]SCH 23390, 0.3 nM

### Control Compound: SCH₂₃₃₉₀

Incubation Conditions: The reactions were carried out in 50 mM TRIS-HCl (pH 7.4) containing 120 mM NaCl, 5 mM KCl, 5 mM MgCl₂, 1 mM EDTA for 60 minutes at 22° C. The reaction was terminated by rapid vacuum filtration onto glass fiber filters. Radioactivity trapped onto the filters was determined and compared to control values in order to ascertain any interactions of test compounds with the cloned dopamine—D₁ binding site.

### Dopamine, D_{2L} Radioligand Binding Assay

### Materials and Methods:

Receptor Source: Human recombinant D_{2L} expressed HET-293 cells

### Radioligand: [³H]Methylspiperone, 0.3 nM

### Control Compound: Butaclamol

Incubation Conditions: The reactions were carried out in 50 mM TRIS-HCl (pH 7.4) containing 120 mM NaCl, 5 mM KCl, 5 mM MgCl₂, 1 mM EDTA for 60 minutes at 22° C. The reaction was terminated by rapid vacuum filtration onto glass fiber filters. Radioactivity trapped onto the filters was determined and compared to control values in order to ascertain any interactions of test compounds with the cloned dopamine—D₂a binding site.

### Dopamine, D_{2S} Radioligand Binding Assay

### Materials and Methods:

Receptor Source: Human recombinant D_{2S} expressed CHO or HEK cells
Radioligand: [³H]Spiperone (20-60 Ci/mmol) or [3H]-7-hydroxy DPAT, 1.0 nM

### Control Compound: Haloperidol or Chlorpromazine

Incubation Conditions: The reactions were carried out in 50 mM TRIS-HCl (pH 7.4) containing 120 mM NaCl, 5 mM KCl, 5 mM MgCl₂, 1 mM EDTA for 60 minutes at 25 C. The reaction was terminated by rapid vacuum filtration onto glass fiber filters. Radioactivity trapped onto the filters was determined and compared to control values in order to ascertain any interactions of test compounds with the cloned dopamine—D₂ short binding site (Literature Reference: Jarvis, K. R. et al. Journal of Receptor Research 1993, 13(1-4), 573-590; Gundlach, A. L. et al. Life Sciences 1984, 35, 1981-1988.)

### Dopamine, D_{4.4} Radioligand Binding Assay

### Materials and Methods:

Receptor Source: Human recombinant D_{2S} expressed CHO cells

### Radioligand: [³H]Spiperone, 0.3 nM, 1.0 nM

### Control Compound: (+)Butaclamol

Incubation Conditions: The reactions were carried out in 50 mM TRIS-HCl (pH 7.4) containing 120 mM NaCl, 5 mM KCl, 5 mM MgCl₂, 1 mM EDTA for 60 minutes at 25 C. The reaction was terminated by rapid vacuum filtration onto glass fiber filters. Radioactivity trapped onto the filters was determined and compared to control values in order to ascertain any interactions of test compounds with the cloned dopamine—D_{4.4} binding site

### Dopamine, D₅ Radioligand Binding Assay

### Materials and Methods:

Receptor Source: Human recombinant D_{2S} expressed GH4 cells

### Radioligand: [3H]SCH 23390, 0.3 nM

### Control Compound: SCH 23390

Incubation Conditions: The reactions were carried out in 50 mM TRIS-HCl (pH 7.4) containing 120 mM NaCl, 5 mM KCl, 5 mM MgCl₂, 1 mM EDTA for 60 minutes at 25° C. The reaction was terminated by rapid vacuum filtration onto glass fiber filters. Radioactivity trapped onto the filters was determined and compared to control values in order to ascertain any interactions of test compounds with the cloned dopamine—D₅ binding site

### Serotonin, 5HT_{1A} Radioligand Binding Assay

### Materials and Methods:

Receptor Source: Human recombinant 5-HT_{1A} expressed mammalian cells

### Radioligand: [³H]-8-OH-DPAT (221 Ci/mmol)

### Control Compound: 8-OH-DPAT

Incubation Conditions: The reactions were carried out in 50 mM TRIS-HCl (pH 7.4) containing 10 mM MgSO₄, 0.5 mM EDTA and 0.1% Ascorbic acid at room temperature for 1 hour. The reaction was terminated by rapid vacuum filtration onto glass fiber filters. Radioactivity trapped onto the filters was determined and compared to control values in order to ascertain any interactions of test compounds with the cloned serotonin—5HT_{1A} binding site (Literature Reference: Hoyer, D. et al. Eur. Journal Pharmacol. 1985, 118, 13-23; Schoeffter, P. and Hoyer, D. Naunyn-Schmiedeberg's Arch. Pharmac. 1989, 340, 135-138)

### Serotonin, 5HT_{2A} Radioligand Binding Assay

### Materials and Methods:

Receptor Source: Human Cortex or Human recombinant 5-HT_{2A} expressed mammalian cells

### Radioligand: [³H]-Ketanserin (60-90 Ci/mmol)

### Control Compound: Ketanserin

Incubation Conditions: The reactions were carried out in 50 mM TRIS-HCl (pH 7.6) at room temperature for 90 minutes. The reaction was terminated by rapid vacuum filtration onto glass fiber filters. Radioactivity trapped onto the filters was determined and compared to control values in order to ascertain any interactions of test compounds with the serotonin—5HT_{2A} binding site (Literature Reference: Leysen, J. E. et al. Mol. Pharmacol. 1982, 21, 301-314; Martin, G. R. and Humphrey, P. P. A. Neuropharmacol. 1994, 33(3/4), 261-273.) Serotonin, 5HT₂B radioligand binding assay

### Materials and Methods:

Receptor Source: Human recombinant 5-HT2B expressed CHO-K1 cells
Radioligand: 1.20 nM [3H] Lysergic acid diethylamide (LSD)

### Control Compound: Ketanserin

Incubation Conditions: The reactions were carried out in 50 mM TRIS-HCl (pH 7.6) at room temperature for 90 minutes. The reaction was terminated by rapid vacuum filtration onto glass fiber filters. Radioactivity trapped onto the filters was determined and compared to control values in order to ascertain any interactions of test compounds with the serotonin—5HT2B binding site

### Serotonin, 5HT₆ Radioligand Binding Assay

### Materials and Methods:

Receptor Source: Human recombinant 5-HT₆ expressed mammalian cells

### Radioligand: [125I] SB258585, 15 nM or [³H]LSD, 2 nM

Control Compound: Methiothepin or serotonin
Incubation Conditions: The reactions were carried out in 50 mM TRIS-HCl (pH 7.4) containing 10 mM MgSO₄, 0.5 mM EDTA and 0.1% Ascorbic acid at room temperature for 1 hour. The reaction was terminated by rapid vacuum filtration onto glass fiber filters. Radioactivity trapped onto the filters was determined and compared to control values in order to ascertain any interactions of test compounds with the cloned serotonin—5HT₆ binding site (Literature Reference: Gonzalo, R., et al., Br. J. Pharmacol., 2006 (148), 1133-1143)

### Serotonin, 5HT₇ Radioligand Binding Assay

### Materials and Methods:

Receptor Source: Human recombinant 5-HT₇ expressed CHO cells
Radioligand: [3H] Lysergic acid diethylamide (LSD), 4 nM

### Control Compound: Serotonin

Incubation Conditions: The reactions were carried out in 50 mM TRIS-HCl (pH 7.6) at room temperature for 90 minutes. The reaction was terminated by rapid vacuum filtration onto glass fiber filters. Radioactivity trapped onto the filters was determined and compared to control values in order to ascertain any interactions of test compounds with the serotonin—5HT₇ binding site
Nicotinic Acetylcholine α₄β₂ (nACh-α₄β₂) Radioligand Binding Assay

### Materials and Methods:

Receptor Source: Human recombinant nACh-α₄β₂ expressed mammalian cells

### Radioligand: [3H] Cytisine, 3.0 nM

### Control Compound: Epibatidine

Incubation Conditions: The reactions were carried out in 120 mM NaCl, 2.5 mM KCl, 50 mM Tris, 1 mM CaCl₂, 1 mM MgCl₂ containing buffer (pH 7.4) for 60 minutes at ambient temperature (37° C.). The reaction was terminated by rapid vacuum filtration onto glass fiber filters. Radioactivity trapped onto the filters was determined and compared to control values in order to ascertain any interactions of test compounds with the cloned nicotinic acetylcholine α₄β₂ (nACh-α₄β₂) binding site

### Serotonin Transporter (SERT) Radioligand Binding Assay

### Materials and Methods:

Receptor Source: Human recombinant H₁ expressed mammalian cells

### Radioligand: [3H] Citalopram, 2.0 nM

### Control Compound: Venlafaxine

Incubation Conditions: The reactions were carried out in 50 mM TRIS-HCl (pH 7.4) containing 120 mM NaCl, 5 mM KCl, 5 mM MgCl₂, 1 mM EDTA for 180 minutes at ambient temperature (37° C.). The reaction was terminated by rapid vacuum filtration onto glass fiber filters.

Radioactivity trapped onto the filters was determined and compared to control values in order to ascertain any interactions of test compounds with the cloned serotonin transporter (SERT) site.

| Compound | Radioligand Binding Assay | Ki (nM) |
|---|---|---|
| A | Dopamine D2S | 0.30 |
| A | Serotonin 5-HT1A | 0.65 |
| A | Serotonin 5-HT2A | 111 |
| B | Dopamine D1 | 100 |
| B | Dopamine D2L | 0.45 |
| B | Dopamine D2S | 0.28 |
| B | Dopamine D3 | 3.7 |
| B | Dopamine D4.4 | 6.0 |
| B | Serotonin 5-HT1A | 1.5 |
| B | Serotonin 5-HT2A | 2.5 |
| B | Serotonin 5-HT2B | 0.19 |
| B | Serotonin 5-HT6 | 51 |
| B | Serotonin 5-HT7 | 2.7 |
| B | Serotonin Transporter (SERT) | 107.1 |
| B | Nicotinic Acetylcholine α4β2 | 36.3 |

### Example 2. Treatment of Signs and Symptoms of Behavioral and Psychological Symptoms in Patients Having Alzheimer's Disease

Objective: This study examines whether the compound of this invention can treat signs and symptoms of agitation in Alzheimer's disease.
Test Compound: Compound B, 6-(4-(4-(2,3-dichlorophenyl)piperazin-1-yl)butoxy)-2H-benzo[b][1,4]oxazin-3(4H)-one hydrochloride, is formulated in the form of liquid, tablet, or capsule.
Placebo contains the same vehicle without the active compound.

### Patent Inclusion Criteria:

Non-institutionalized patients with a diagnosis of Alzheimer's disease as defined by Diagnostic and Statistical Manual of Mental Disorders—Fourth Edition (DSM-IV) criteria with symptoms of delusions or hallucinations, which have been present, at least intermittently for one month or longer
Mini Mental State Examination (MMSE) score of 6 to 24 points
Patients capable of self-locomotion or locomotion with the aid of an assistive device Patent Exclusion Criteria:
Patients with an Axis I (DSM IV) diagnosis of: delirium, amnestic disorders, bipolar disorder, schizophrenia or schizoaffective disorder, mood disorder with psychotic features
Patients with reversible causes of dementia
Patients with psychotic symptoms continuously present since prior to the onset of the symptoms of dementia
Patients with psychotic symptoms that are better accounted for by another general medical condition or by direct physiological effects of a substance
Patients with a current major depressive episode with psychotic symptoms of hallucinations or delusions
Patients with a diagnosis of dementia related to infection with the human immunodeficiency virus
Patients with substance-induced persistent dementia
Patients with dementia due to vascular causes, multi-infarct, head trauma, Pick's disease, Parkinson's disease, frontal or temporal dementia, Lewy body dementia, or any specific non-Alzheimer's type dementia
Patients with seizure disorders
Patients who have been refractory to neuroleptics used to treat psychotic symptoms in the past when treated for an adequate period with a therapeutic dose,
Patients who have met DSM-IV criteria for any significant substance use disorder within the 6 months prior to the start of screening

### Methodology:

This is placebo-controlled, double-blind treatment clinical activity study.

Treatment Duration: 1 week pretrial washout, 6 weeks of drug treatment, and 1 week post trial re-stabilization.

A total of 20-120 patients are enrolled; about ¾ of the patients are treated with Compound B, and ¼ of the patients are treated with placebo. The test compounds and the placebo are delivered either by oral administration or by transdermal patch for 8 weeks.

For oral administration, patients take 0.5-100 mg of test compound or placebo once a day.

For transdermal administration, doses that achieve similar blood concentration as that of effective oral doses are given to patients. The patches are replaced every week or every two weeks.

Patients are monitored by the treating psychiatrist. Study assessments are administered at designated time points.

### Criteria for Evaluation:

### Primary Outcome Measures:

Change From Baseline in Neuropsychiatric Inventory (NPI) Psychosis Subscale Score at Week 6. The NPI is a questionnaire that quantifies behavioral changes in dementia. For each of 12 behavioral domains there are 4 scores: Frequency (scale: 1=occasionally to 4=very frequently), Severity (scale: 1=Mild to 3=Severe), Total (frequency×severity), Caregiver distress (scale: 0=not at all distressing to 5=extremely distressing). The NPI Psychosis Subscale consists of the two domains of Delusions and Hallucinations, calculated by adding the Individual Item Scores, to yield a possible total score of 0 to 24. Lower score=less severity. A negative change score from baseline indicates improvement.

### Secondary Outcome Measures:

Participants Who Demonstrated a ≧50% Decrease From Baseline to Endpoint in the NPI Psychosis Subscale Score in Acute Phase week 6
Change From Baseline in NPI Psychosis Subscale Caregiver Distress Score in Acute Phase week 6
Change From Baseline in Clinical Global Impression (CGI) Severity of Illness Score in Acute Phase. Time Frame: Baseline (Day 0) through Weeks 1, 2, 3, 4, 6, 8.
The CGI rating scale, which measures symptom severity, treatment response and the efficacy of treatments, is used in clinical studies on mental disorders.
CGI Severity scale is a 7-point scale that requires the clinician to rate the severity of the illness at the time of assessment, relative to the clinician's past experience with participants who have the same diagnosis. The assessment is based on severity of mental illness at the time of rating, 0=not assessed, 1=normal, 2=borderline mentally ill; 3=mildly ill; 4=moderately ill; 5=markedly ill; 6=severely ill; or 7=extremely ill.
CGI Improvement Score in Acute Phase Weeks 1, 2, 3, 4, 6, and 8
Change From Baseline in Brief Psychiatric Rating Scale (BPRS). Total Score in Time Frame: Baseline (Day 0), Weeks 1, 2, 3, 4, 6, and 8
Change From Baseline in Mini Mental State Examination (MMSE). Total Score in Time Frame: Baseline (Day 0), Week 8. The MMSE is a screening test for cognitive dysfunction. The test consists of five sections (orientation, registration, attention-calculation, recall, and language). It is a 19 item scale, the total score can range from 0 to 30, with a higher score indicating better function. A positive change score indicates improvement from baseline.
Cognition Test Battery change form baseline

### Safety Measures:

Vital signs, laboratory, ECG, physical examination

While the invention has been particularly shown and described with reference to a preferred embodiment and various alternate embodiments, it will be understood by persons skilled in the relevant art that various changes in form and details can be made therein without departing from the scope of the invention. All printed patents and publications referred to in this application are hereby incorporated herein in their entirety by this reference.

What is claimed is:

1. A method of treating behavioral and psychological symptoms in a patient having Alzheimer's disease, comprising administering to a patient in need thereof an effective amount of a compound of Formula I: 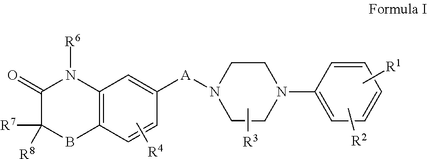
or a pharmaceutically acceptable salt, isomer, racemate, or diastereomeric mixture thereof, wherein:
A is —O—(CH₂)ₙ—, —(CH₂)ₙ—, —S—(CH₂)ₙ—, —NH—(CH₂)ₙ—, —CH₂—O—(CH₂)ₙ—, —(CH₂)ₙ—O—CH₂—CH₂—, —CH₂—S—(CH₂)ₙ—, —NH—C(O)—(CH₂)ₙ—, —CH₂—NH—C(O)—(CH₂)ₙ—, —CH₂—C(O)—NH—(CH₂)ₙ—, or —(CH₂)ₙ—C(O)—NH—CH₂—CH₂—, wherein n is an integer from 1 to 7;
B is O, S, S(O)(O), or NR⁵; and
R¹, R², R³, R⁴, R⁶, R⁷, and R⁸ are independently hydrogen, alkyl, substituted alkyl, aryl, substituted aryl, arylalkyl, substituted arylalkyl, cycloalkyl, substituted cycloalkyl, alkoxy, alkoxycarbonyl, alkylsulfinyl, alkylsulfonyl, alkylthio, amino, alkylamino, dialkylamino, arylalkoxy, carboxy, carbamoyl, carbamate, carbonate, cyano, halogen, or hydroxy; wherein the hydrogen of R¹, R², R³, R⁴, R⁶, R⁷ and R⁸ and A are optionally substituted with ²H (deuterium).

2. The method according to claim 1, wherein A is —O—(CH₂)ₙ—.

3. The method according to claim 1, wherein A is —(CH₂)ₙ—.

4. The method according to claim 1, wherein A is —NH—C(O)—(CH₂)ₙ—, —CH₂—NH—C(O)—(CH₂)ₙ—, —CH₂—C(O)—NH—(CH₂)ₙ—, or —(CH₂)ₙ—C(O)—NH—CH₂—CH₂—.

5. The method according to claim 1, wherein B is O.

6. The method according to claim 1, wherein R³, R⁴, R⁶, R⁷, and R⁸ are hydrogen.

7. The method according to claim 6, wherein R¹ and R² are independently H, halogen, or alkoxy.

8. The method according to claim 7, wherein R¹ is H, and R² is methoxy.

9. The method according to claim 7, wherein R¹ and R² are chloro.

10. The method according to claim 1, wherein A is —O—(CH₂)ₙ—; B is O, and R³, R⁴, R⁶, R⁷, and R⁸ are independently hydrogen or alkyl.

11. The method according to claim 1, wherein the compound is 6-(4-(4-(2,3-dichlorophenyl)piperazin-1-yl)butoxy)-2H-benzo[b][1,4]oxazin-3(-4H)-one, or its hydrochloride salt thereof.

12. The method according to claim 1, wherein the compound is 6-(4-(4-(2-methoxyphenyl)piperazin-1-yl)butoxy)-2H-benzo[b][1,4]oxazin-3(-4H)-one, or its hydrochloride salt thereof.

13. The method according to claim 1, wherein the compound is administered in a pharmaceutical composition comprising a pharmaceutically acceptable carrier, excipient, or diluent.

14. The method according to claim 1, wherein the compound is orally administered.

15. The method according to claim 1, which treats dementia.

16. The method according to claim 1, which treats memory impairment and/or cognitive impairment in the patient.

17. The method according to claim 1, which treats agitation in the patient.

18. The method according to claim 1, which treats psychosis in the patient.

19. The method according to claim 1, which treats depression in the patient.

20. The method according to claim 1, which treats mood swing in the patient.
